(19) United States
(12) Patent Application Publication
(10) Pub. No.: US 2011/0004304 A1
(43) Pub. Date: Jan. 6, 2011

(54) CULTURING RETINAL CELLS AND TISSUES

(76) Inventors: Sarah L. Tao, Cambridge, MA (US); Stephen Redenti, Boston, MA (US); Sonal Sodha, Potomac, MD (US); Jeffrey T. Borenstein, Newton, MA (US); Michael Young, Ipswich, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/727,881

(22) Filed: Mar. 19, 2010

Related U.S. Application Data
(60) Provisional application No. 61/162,106, filed on Mar. 20, 2009.
(60) Provisional application No. 61/216,947, filed on May 21, 2009.

(51) Int. Cl. A61F 2/14; C12M 3/00; C12N 5/00; A61K 47/06; A61K 35/44; A61K 9/00; A61P 27/02

(52) U.S. Cl. 623/6.63; 435/297.2; 435/377; 435/373; 514/772; 424/93.7; 424/400

(57) ABSTRACT

Disclosed are various methods and bioreactor devices for culturing retinal cells and/or tissues. The bioreactor devices may, in certain embodiments, include a microchannel network, a scaffold for culturing neuroretinal cells, and a porous membrane separating the microchannel network from the scaffold.

### CROSS-REFERENCE TO RELATED APPLICATIONS

This application claims priority to and the benefit of U.S. Provisional Patent Applications No. 61/162,106, filed Mar. 20, 2009, and No. 61/216,947, filed May 21, 2009, which are hereby incorporated herein by reference in their entirety.

### FIELD OF THE INVENTION

In various embodiments, the present invention relates to device, systems, and methods for cell culturing and tissue engineering, and, in particular, to bioreactor structures for culturing retinal cells and tissues and methods of using the same.

### BACKGROUND

The human retina is a complex, delicate structure with an extremely limited capacity for self-repair. Unfortunately, this delicate, yet important neural tissue is prone to a range of diseases and injuries, many of which result in significant visual disability, in the most severe cases total blindness.

The neural retina depends on stringently controlled homeostasis to maintain its function. FIG. 1 schematically illustrates the neural retina 102, the outer blood-retina barrier (oBRB) 104, and the choriocapillaris (CC) 106, a capillary layer of the choroid 107 that supplies blood to the retina. The oBRB 104 is comprised of Bruch's membrane (BM) 108 and a layer of pigmented epithelial cells forming the retinal pigment epithelium (RPE) 110. An intact and undamaged BM is essential for the growth and attachment of RPE cells.

Two diseases affecting the retina, age-related macular degeneration (AMD) and retinitis pigmentosa, typically result in the degeneration of photoreceptor cells. In the United States, AMD is the leading cause of blindness among the elderly. Typically, the major causes of severe visual loss among patients with AMD are choroidal neovascularization (CNV), which may result from defects in Bruch's membrane, and atrophy of the RPE. There is generally no treatment for atrophy, and pathway-based pharmacological therapy for CNVs (e.g., the administration of bevacizumab and ranibizumab, which block the action of vascular endothelial growth factor) typically results in moderate visual improvement in only a minority (i.e., less than 30%) of patients, so the need for an alternative approach remains urgent.

In addition to diseases, a frequent cause of damage to the retina are blast injuries, resulting, for example, from detonation of improvised explosive devises (IEDs). Blast injuries constitute about 80% of injuries to U.S. troops returning from Iraq. While advances in body armor technology have saved the lives of numerous soldiers through protection of vital organs and the skull, the eyes remain relatively vulnerable to injury in an IED attack, even with the use of protective equipment. During a traumatic blast, damage to the posterior segment of the eye can occur from direct trauma of a penetrating projectile path, as well as from indirect trauma by shock waves and compressive forces transmitted to the globe of the eye. Injury is caused by the pressure differential across delicate membranes, which implodes the affected tissues. Bruch's membrane, which is less elastic than the retina and weaker in strength than the adjacent sclera, is particularly susceptible to traumatic rupture. When the membrane is ruptured, the CC and the RPE also tear, often extending injury through the full thickness of the retina. Trauma to the oBRB elicits a natural wound healing response that is both inflammatory and angiogenic in nature, further disrupting RPE architecture and inhibiting its ability to self-repair. Loss of oBRB structure can directly lead to photoreceptor death in the neural retina and atrophy of the optic nerve, both of which are irreversible. In a clinical situation in which the local laminar cytoarchitecture has been disrupted and the resident cell population depleted, a strategy is needed to replace lost cells and induce re-organization of the ocular structure.

There is continuous demand in ophthalmic research for the investigation of new treatment modalities of neuroretinal disease, such as pharmaceutical agents, cell-based therapy, or transplant therapy. Cell culture models are often used as a first step in investigating new treatment modalities. Most retinal cell culture models are based on proliferating cell types cultivated under conventional static culture conditions, utilizing tissue culture plastic or transwell inserts. Few cell culture models have used co-culture (e.g., RPE cells with vascular endothelial cells), and generally no cell culture models have been able to establish an in vitro model of the native, stratified layers resembling the oBRB. Simple static co-culture models are limited in several ways, including, for example, that endothelial tubes with a lumen typically cannot be formed without a tridimensional collagen matrix present, seeding of RPE cells at low densities typically prevents formation of a confluent monolayer, mixed cultures typically do not resemble the physiological interfaces at the back of the eye, and manipulations of individual cell populations typically cannot be easily achieved.

In an alternative approach, a rotary bioreactor has been utilized to form cell aggregates of three-dimensional retinal cell structures. The use of a culture rotary system typically promotes cell-to-cell interaction, while retaining a spectrum of differentiation capability. However, the cells typically have to be maintained on cytodex beads that are unsuitable for transplant purposes. While improvements of the rotary culture approach have allowed spheroid cell aggregates, these spheroids typically do not form the stratified sheet-like layers of the inherent retina and, upon bolus injections into a host eye, do not integrate well with the native tissue.

One of the most promising therapies for vision loss due to irreversible damage to the retina, whether resulting from disease or injury, is transplantation of tissue-engineered constructs. A tissue-engineering strategy may avoid various problems associated with injection-based cell delivery to the subretinal space, such as immediate reflux at the time of injection and/or massive cell death due to shear forces involved in the process. Further, tissue engineering offers, in principle, the opportunity to recreate the complex cytoarchitecture of the retina, which is particularly important when multiple neuroretinal layers have been lost or disrupted.

Various polymer scaffolds for cell delivery have been developed. For example, irregular, porous bulk scaffolds have been used to deliver large numbers of cells. However, in this approach, the cells are not in direct contact with each other or the surrounding environment, and therefore do not integrate with the surrounding tissue until they have migrated into the host retina. In an alternative approach, two-dimensional cell culture platforms, such as polymer sheets with through pores or films of nanowires, have been used. While these scaffolds facilitate direct contact of the cells with the host retina environment, the total number of cells that can be delivered is typically limited, and the cells are exposed to a high degree of shear during the transplant process.

Accordingly, it is desirable to establish improved in-vitro models of retinal cell co-cultures that more closely resemble the native, stratified layers of the outer retina. Further, there is a need for transplantable structures and devices that facilitate the culturing and differentiation of large numbers of retinal cells, as well as their delivery to and integration into a host retina.

### SUMMARY

Described herein are various embodiments of bioreactor devices for culturing neuroretinal cells and/or tissues. In general, the bioreactor devices are designed to mimic the architecture of one or more layers of the retina, including, e.g., the neural retina, choriocapillaris, and/or choroid-neuroretina interface (which includes Bruch's membrane). The devices may, for example, comprise a polymer-based scaffold that is topographically and/or chemically structured at a micrometer or nanometer scale so as to facilitate the physiologically realistic spatial organization and assembly of cells seeded in the scaffold. Further, the devices may include perfused microfluidic structures for supplying the cells with oxygen and nutrients, as well as with biochemical or fluid-mechanical cues for cell differentiation.

In one embodiment, the bioreactor device is based on the reconstruction of stratified neuroretinal tissue in a three-dimensional (3D) configuration. Such a device may include an interdigitated network of microchannels (hereinafter also referred to as a “plexus”) mimicking the choriocapillaris, an artificial Bruch's membrane for RPE cell culture, and/or a chamber or microstructured scaffold for the introduction of neuroretinal cells or cells capable of differentiating into neuroretinal cells (such as retinal progenitor cells (RPCs) or stem cells). The microchannel network may be endothelialized, i.e., contain vascular endothelial cells. In one embodiment, the structured scaffold includes a cage-like structure for holding cells, which may interact with the RPE through smaller pores in one surface, and with a host retinal tissue through larger pores in the opposite surface.

Bioreactor devices in accordance with various embodiments provide in-vitro models of the neuroretina and/or retinal blood barrier that may be used to study the dynamic functions of the retina and mechanisms of retinal damage and disease, as well as to test substances designed for intra-ocular application, such as drug candidates. For example, the RPE cells, artificial Bruch's membrane, and artificial plexus, which collectively mimic the retinal blood barrier, may be utilized as a model for neovascularization. Further, cells cultured in the bioreactor may be utilized in cell-based therapy for diseased or damaged retina tissue. Alternatively or additionally, the bioreactor structures may be biodegradable, and serve to engineer retinal tissue which, as a whole, may be transplanted into a patient's eye to replace damaged tissue.

Accordingly, in a first aspect, embodiments of the invention provide a bioreactor for culturing retinal tissue. The bioreactor includes a first polymer layer defining a network of microchannels, a second polymer layer forming a scaffold, and a porous thin-film membrane separating the first polymer layer from the second polymer layer. The membrane is coated with retinal pigment epithelial cells on a surface facing the second polymer layer.

One or more of the following features may be included. The membrane may be made of or include a polymer material (e.g., a polymer having a Young's modulus of at least 0.1 MPa) and may have a thickness in the range from about 2 μm to about 6 μm. Pores of the membrane may have diameters of less than 500 nm. The membrane may be characterized by a diffusivity in the range from 200 μg/mm² per day to 300 μg/mm² per day. An inner surface of the scaffold may be topographically and/or chemically patterned. Further, the scaffold may be microstructured so as to provide contact guidance for spatial cell organization. Alternatively, the scaffold may form a hollow cell culture chamber. The network of microchannels may form an artificial plexus.

In some embodiments, the bioreactor includes RPCs and/or stem cells (i.e., embryonic or adult stem cells, such as, e.g., induced pluripotent stem cells) seeded in the scaffold. Further, the bioreactor may contain cell culture media in the scaffold and/or the microchannel network. The bioreactor may also have means for perfusing the microchannel network and/or the scaffold, as well as means for controlling a pressure in at least one of the microchannel network or the scaffold.

In a second aspect, various embodiments of the invention are directed to a bioreactor including a first polymer layer defining an artificial plexus, a second polymer layer forming a microstructured scaffold, and a porous thin-film polymer membrane separating the first polymer layer from the second polymer layer. The microstructured scaffold may include polymer posts or, alternatively, pores arranged at the vertices of a lattice. In some embodiments, the pores have a hexagonal shape, or a modified hexagonal shape wherein the straight edges of a hexagon are replace by curved convex or concave edges. Clusters of pores (e.g., round pores) may be arranged hexagonally. (Six or more pores that are hexagonally arranged in a way that they overlap would result in a modified hexagonal shape with convex edges.) The microstructured scaffold may include a hard polymer (e.g., polycaprolactone) structure forming pores that are filled with a soft polymer (e.g., hyaluronic acid). In certain embodiments, the microstructured scaffold includes top, middle, and bottom layers, the top and bottom layers having through pores, and the three layers together defining a cage structure for holding cells.

In a third aspect, a method for culturing retinal tissue in a bioreactor such as any of the bioreactors described above is provided. The method involves seeding RPCs or stem cells in the scaffold, perfusing the network of microchannels with a fluid suitable for cell culture, and initiating cell differentiation of the retinal progenitor cells. Further, the method may include seeding RPE cells on a surface of the membrane facing the second polymer layer, and/or seeding vascular endothelial cells in the microchannels. Cell differentiation may be induced by RPE cells. Alternatively or additionally, cell differentiation may be initiated by supplying a neurotrophic factor or cell signaling molecule (e.g., via the cell culture fluid). The bioreactor may be transplanted into a patient's retina.

In a fourth aspect, the invention provides, in various embodiments, a cell-delivery device including a polymer scaffold that defines one or more cages for housing cells. The scaffold features a first set of pores on a first surface and a second set of pores that are smaller than the pores of the first set on a second surface. For example, the pores of the first set may have diameters between about 5 μm and about 70 μm, and the pores of the second set may have diameters between about 1 μm and about 20 μm. The pores of the first and second sets are in fluidic communication with the cage(s), which may have a lateral diameter (i.e., a largest dimension parallel to the first and second surfaces) in the range from about 150 μm to about 300 μm.

One or more of the following features may be included. Clusters of pores of the first set may be hexagonally packed, for example, in groups of seven pores. If the distance between centers of the seven pores is smaller than the pore diameter, the seven pores merge into one larger pore of modified hexagonal shape. The pores of the first and/or second sets may be arranged at the vertices of a lattice (e.g., a square lattice or a hexagonal lattice). The polymer scaffold may me made of or include a biodegradable material, and the cage(s) may be filled with a soft polymer material. Therapeutic molecules may be incorporated in the polymer scaffold, e.g., by absorption, embedding, encapsulation, nanoparticle incorporation, hydrogel delivery, or conjugation to the scaffold surface. Further, cells (e.g., retinal cells, retinal progenitor cells, and/or stem cells) may be housed in the scaffold. In various embodiments, the polymer scaffold is implantable into a patient's retina and the pores permit ingress and egress of nutrients and/or regulators.

In certain embodiments, the polymer scaffold is formed of three polymer layers: a first layer defining the side walls of the cage(s), a second polymer layer defining the first set of pores, and a third polymer layer defining the second set of pores. The second layer is bonded to a first surface of the first layer, and the third layer is bonded to a second surface of the first layer. Each of the three polymer layers may have a thickness between about 1 μm to about 20 μm.

In a fifth aspect, embodiments of the invention provide a bioreactor device for mimicking the architecture of one or more layers of the retina. The device includes a polymer-based scaffold structured at a micrometer or lower scale (e.g., topographically or chemically) so as to facilitate physiologically functional spatial organization and assembly of cells seeded. Further, it includes a microfluidic structure for supplying the cells with biochemical materials such as, e.g., oxygen and/or nutrients. The microfluidic structure may facilitate transmission of biochemical or fluid-mechanical cues for cell differentiation to the cells.

These and other objects, along with advantages and features of embodiments of the present invention herein disclosed, will become more apparent through reference to the following description, the figures, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic drawing illustrating the neural retina, outer blood-retina barrier, and choriocapillaris of an eye;

FIGS. 2A and 2B are schematic perspective views of bioreactor devices in accordance with various embodiments;

FIGS. 3A-3C are schematic top views of plexus structures in accordance with various embodiments;

FIG. 4 is a schematic top view of a membrane in accordance with various embodiments;

FIGS. 5A-5B are schematic drawings illustrating the arrangement of photoreceptor cells in the neuroretina;

FIGS. 5C-5D are perspective views of portions of microstructured scaffolds in accordance with various embodiments;

FIG. 6 is a schematic perspective view of a cage-like microstructured scaffold in accordance with one embodiment; and

FIGS. 7A-7D are schematic top views of various layers of a cage-like microstructured scaffold in accordance with one embodiment.

### DETAILED DESCRIPTION

### 1. Bioreactor Structures

In general, the present invention provides, in various embodiments, bioreactor devices for retinal cell and tissue culturing. In certain embodiments, the devices are microfluidic systems that include (i) a layer defining a network of microchannels, or plexus, that mimics the choriocapillaris, (ii) an apical chamber or microstructured scaffold for culturing neuroretinal cells, and (iii) a porous, thin-film membrane mimicking the transport properties of Bruch's membrane, sandwiched between the plexus layer and the cell culture chamber or scaffold. Further, the system may, optionally, include mechanical and/or electronic control elements for setting and adjusting convective flow and hydrodynamic force.

The microchannel layer and the chamber or scaffold are typically formed of any of a variety of non-degradable or degradable polymer materials, although they alternatively may be made of non-polymer materials (e.g., glass or ceramics) for certain applications, in particular those which do not require implantation in the eye. Suitable non-degradable polymer materials include polystyrene, polydimethylsiloxane (PDMS), polycarbonate, poly(methyl methacrylate), and polyurethane. For tissue engineering applications, the use of biodegradable and/or biocompatible materials, such as polyglycerol sebacate, polyesteramide, polyoctanediol citrate, polydiol citrate, silk fibroin, polycaprolactone, poly(lactic acid), poly(glycolic acid), poly(lactide-co-glycolide), poly(lactide-ci-caprolactone), poly(hydroxyl butyrate), or biopolymers/natural polymers (such as proteins, gels, or extracellular matrix) may be advantageous. Further, in certain embodiments, a harder (i.e., less elastic) polymer defining the walls of the culture chamber or the backbone of the scaffold may be combined with a softer polymer, such as collagen, matrigel, or another gel, filling the openings. In various embodiments, the harder polymer has an elastic modulus in the range from about 0.1 to about 1 kPa, whereas the softer polymer has a modulus exceeding 1 kPa. The thin-film membrane may be fabricated from any number of biodegradable or non-degradable polymer materials or, alternatively, from non-polymer materials such as, e.g., aluminum oxide or silicon oxide.

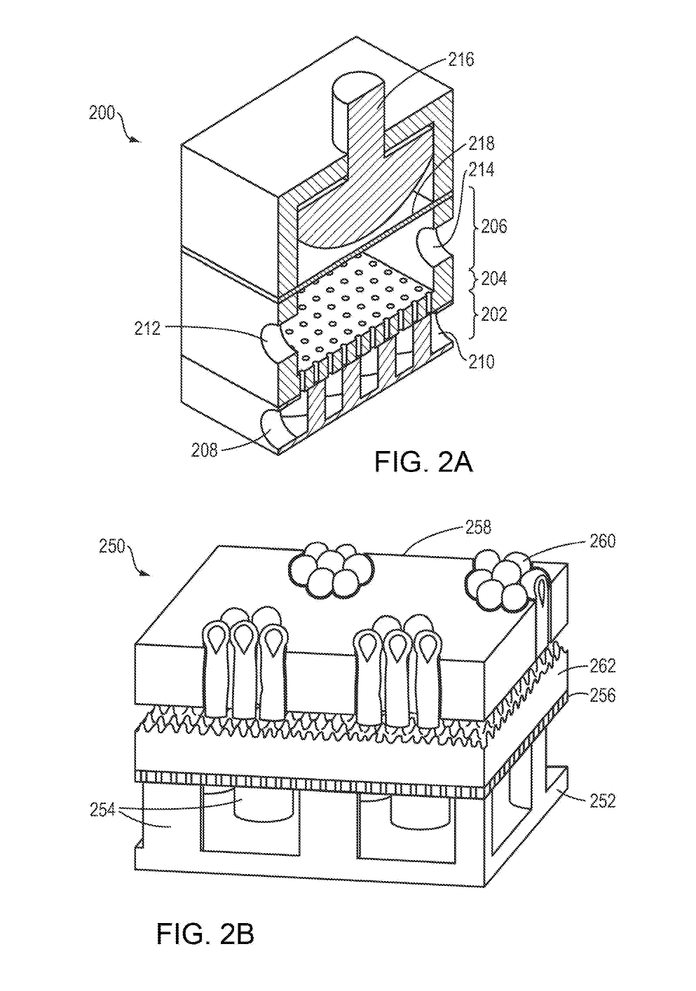

FIGS. 2A and 2B illustrate two exemplary bioreactor devices in perspective views. More specifically, FIG. 2A depicts a bioreactor device 200 including an artificial plexus layer 202, a thin-film membrane 204, and a culture chamber 206 for neuroretinal cell culture and differentiation. One or more fluid inlets 208 and outlets 210 facilitate perfusing the artificial plexus 202 with cell culture medium, water, buffer solution, blood components, or whole blood. The device 200 may be integrated with standard means for controlling flow through the plexus 202, including, for example, a pump and valves, as well as piping that connects the inlet 208 and outlet 210 to the fluidic components exterior to the cell culture construct. In some embodiments, choroidal endothelial cells (not shown) are seeded in the microchannels of layer 202 to create an endothelialized network mimicking the choriocapillaris plexus, and to impart homeostatic balance between molecular interactions of the neural retina, RPE, and vasculature which occurs in the eye.

The culture chamber 206 contains, when in use, RPCs and/or stem cells, and/or differentiated neuroretinal cells. In preferred embodiments, an artificial microstructured scaffold or, alternatively, isolated natural retina tissue, is placed in the culture chamber to facilitate the spatial organization and polarization of the cultured cells. The chamber 206 may be filled with pre-conditioned media. Further, like the plexus 202, the chamber 206 may be continuously perfused via one or more inlets 212 and outlets 214. In some embodiments, the chamber 206 is connected to an additional microfluidic network circuit that mimics the retinal vasculature. Alternatively, the chamber 206 may be left open for bulk transport. The device 200 may further include a pneumatic system that can be used to control the hydrodynamic pressure, e.g., to simulate the intraocular pressure in vivo. The pneumatic system may include a compression post 216 that deflects an elastomeric silicone diaphragm 218 that, in turn, transfers the pressure to the fluid and/or scaffold in the culture chamber 206.

FIG. 2B illustrates a portion of a bioreactor device 250 in use. The device includes, in addition to a plexus layer 252 and a porous membrane 256, a microstructured scaffold 258. The scaffold 258 has pores that house vertically aligned groups of cells 260, e.g. RPCs. Grooves in the pores promote migration and elongation of the cells in the direction of the pores. The membrane 256 is coated with a monolayer of RPE cells 262. To leave room for the RPE, the polymer scaffold 258 is spaced apart from the membrane 256. At the side walls of the device 250 (not shown), the scaffold 258 may reach down to the membrane 256, facilitating bonding between the membrane and scaffold. The structure 250 may have a height in the range from about 70 μm to about 200 μm, comprising a height of the plexus layer of about 35 μm, a membrane thickness of a few micrometers, and a height of the cell culture chamber of about 50 μm.

Bioreactor devices in accordance with certain embodiments may feature various combinations of components and characteristics of the devices 200, 250 described above with respect to FIGS. 2A and 2B. In the following sections, further embodiments of the plexus, membrane, and microstructured scaffold are described in more detail.

### 1.1 Plexus

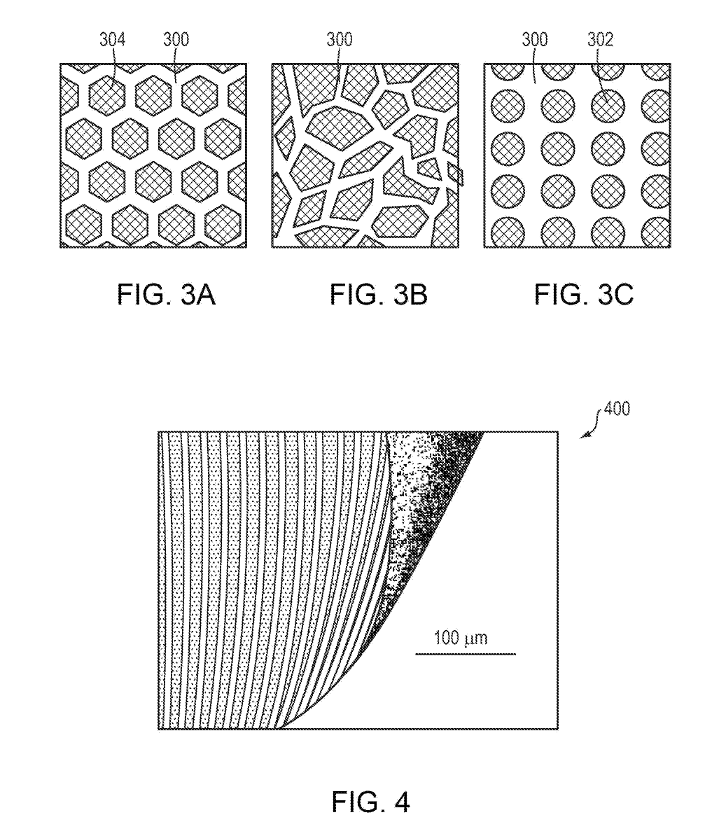

The choriocapillaris is a plexus, i.e., a network of randomly oriented and complexly interconnected capillaries. This microvascular architecture is unique to zones of gas exchange and filtration, and is distinguished from the bifurcating, tree-like vasculatures found in other organs (as well as in other layers of the eye). To model the choriocapillaris with an artificial polymer layer, the plexus may be approximated by a lattice-like network of microchannels 300. For example, the channels 300 may form the edges of a honeycomb lattice, as illustrated in FIG. 3A. Alternatively, to further capture the random orientations of the capillaries in vivo, the network design may feature a random distribution of channel section lengths and angles at interconnections, as illustrated in FIG. 3B. As shown in FIGS. 3A and 3B, the channels 300 may have substantially uniform dimensions along their lengths. However, this need not always be the case. For example, in FIG. 3C, a channel network is shown which is defined by round polymer posts 302 arranged at vertices of a square lattice. Round posts 302 (as opposed to, e.g., hexagonal posts 304 as illustrated in FIG. 3A) inherently result in variable width of the resulting channels 300. Since it is often difficult to move fluid through narrow channels without clogging, the use of posts may provide a better fluidic pathway.

The channel cross sections may have polygonal (e.g., rectangular) or rounded shapes, and dimensions on the order of several micrometers. Specific geometric and size parameters of the network may be chosen (based, for example, on numerical simulations) to achieve desired flow velocities or other fluid-mechanical parameters in the network. For example, in one embodiment, the channels have a rectangular cross section and are 7.5 μm tall and 15 μm wide, resulting in a velocity of the medium of approximately 2 mm/sec when endothelialized. Inlets and outlets may be designed as precapillary feeding arteriolar (e.g., 7.5 μm diameter) and venular (e.g., 15 μm diameter) vessels, which are connected and attached to a syringe pump. The microchannels may be continuously perfused with culture media to maintain the neuroretinal cells in the scaffold via diffusion of nutrients and oxygen across the artificial RPE-Bruch's membrane. The network may further be utilized to introduce test compounds as well as sample media for analysis.

In bioreactor devices designed to model choroidal neovascularization, a parallel layer defining a miniature version of the choriocapillaris plexus, with scaled-down dimensions, can be integrated in the device to result in a significantly higher resistance to fluid flow and a flow velocity of approximately 1 mm/sec. Channels of the neovascularization layer and the choriocapillaris layer may be fluidically connected by capillary-dimensioned vessel through-holes, which also penetrate the artificial Bruch's membrane via prefabricated micro-hole openings.

### 1.2 Porous, Thin-Film Membrane

The thin-film membranes may, generally, be fabricated from a polymer material. The particular choice of material may depend on the application and desired properties of the membrane. In order to be a viable substrate for the retinal epithelium, a membrane designed for transplant into the eye is preferably biocompatible and possesses properties comparable with those associated with the physiological RPE-Bruch's membrane. Consequently, in one embodiment, the custom membrane is smooth (i.e., has a no surface topography on a scale exceeding a few nanometers), and is of similar thickness (e.g., 2-6 μm thick) and diffusivity (e.g., 250 μg/mm² per/day) as a biological Bruch's membrane. Further, in preferred embodiments, the membrane is stronger and/or more elastic than the surrounding natural tissues, having a Young's modulus in the range from about 0.1 MPa to about 500 MPa (compare Young's elastic modulus of the retina=1×10⁵ Pa, Young's elastic modulus of the choroid=10×10⁵ Pa).

The diffusivity of the membrane may be controlled by the size and density of the pores. Pore diameters are typically below 500 nm, to avoid transmigration of endothelial or other cells from the plexus layer into the apical chamber or scaffold, where these cells could otherwise induce an inflammatory response. In one embodiment, the pores have diameters in the range from about 100 nm to about 1 μm and a density in the range from about 1×10³ to about 1×10⁸ pores per mm², resulting in a diffusivity in the range from about 1×10⁻⁵ to about 1×10⁻³ cm/s.

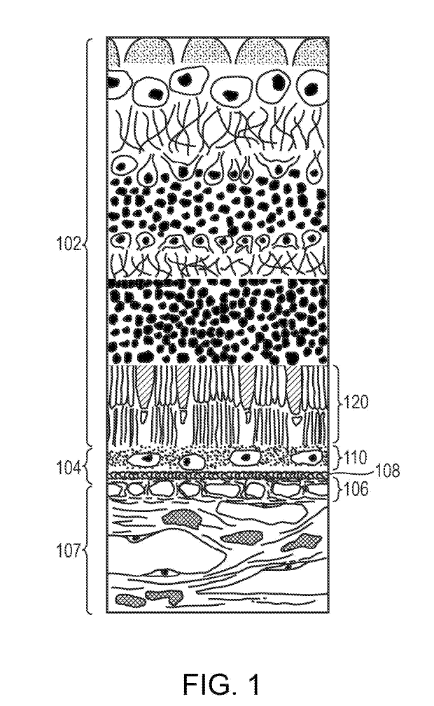

Utilizing microfabrication techniques, the thin-film membranes may be custom-tailored in terms of topography, porosity, and chemistry. For example, to facilitate RPE attachment to the membrane, the apical membrane side may be oxygen-plasma-treated or chemically functionalized, e.g., with laminin, fibronectin, vitronectin, RGD (arginine-glycine-aspartate) or other protein sequences. The membranes may be modified on both sides in order to direct cell interaction with both the RPE cells and the plexus. For example, a grooved pattern may be used to induce elongation and parallel alignment of endothelial cells, whereas a polygonal architecture may be used to aid in formation of RPE cells with distinct shape and size. Further, the apical membrane side may be tailored for functional RPE monolayer development, while the opposing side may be structured to prevent transmigration of endothelial cells in neovascularization models. FIG. 1B depicts an exemplary polymer thin-film membrane 400 structured on two sides.

### 1.3 Microstructured Scaffold

The upper compartment of the bioreactor construct may be utilized as a culture chamber for RPE on the artificial membrane, as well as for the differentiation of RPCs, multipotent retinal stem cells, or pluripotent stem cells into functional neuroretinal cells, such as photoreceptors (i.e., rods and cones) based on a biomimetic architectural topography. In various embodiments, therefore, the upper compartment is comprised of or houses a scaffold of micro- and/or nano-structures that is based on the physiological architecture of the neuroretina. The scaffold may be formed of a network of polymer posts or walls, which leave open spaces for cell attachment and organization. Alternatively, the scaffold may include or essentially consist of a polymer layer with regularly arranged micro-pores that can house individual cells or groups of cells, and facilitate both the polarization (i.e., vertical alignment) of the cells and their interaction with the retinal tissues above and below. The pores may have any of a variety of shapes; they may, for example, be round or polygonal. Further, they may be evenly distributed, e.g., at the vertices of a regular lattice, or clustered.

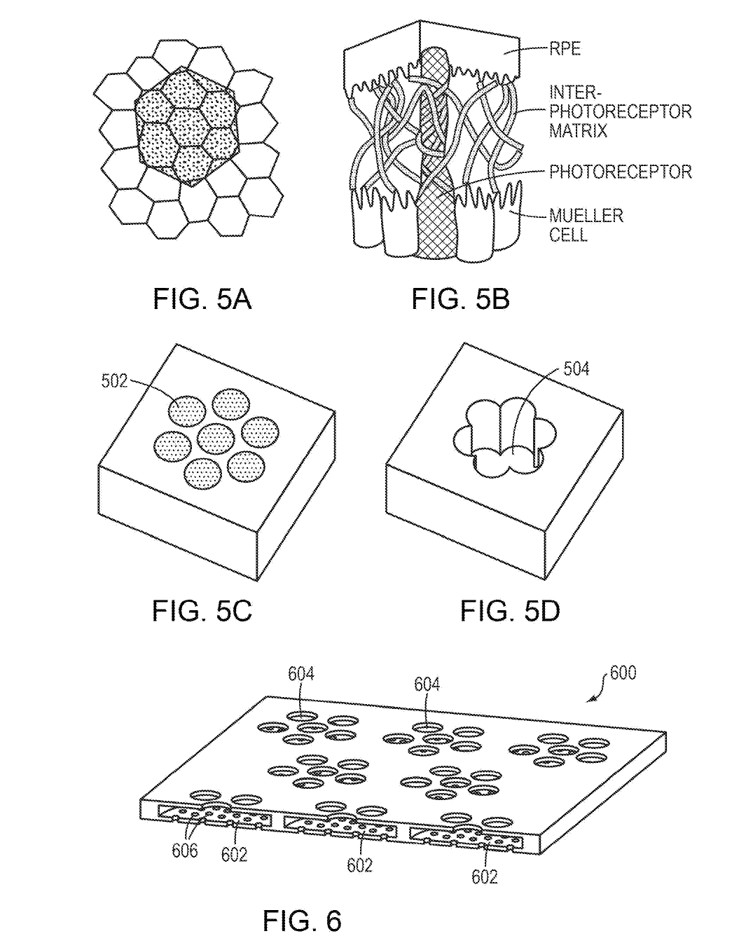

In the human retina, the inner segments of both rods and cones typically exhibit a hexagonal form and are regularly arranged in an approximate honeycomb fashion, i.e., each cone or rod is surrounded by six other cones or rods, respectively. This dense hexagonal packing is schematically illustrated in FIG. 5A. The integration of a photoreceptor into the extracellular, interphotoreceptor matrix, which spans the space between the RPE and the outer limiting membrane of the retina, formed by the Mueller cells, is shown in FIG. 5B. To mimic the hexagonal arrangement of photoreceptors, the scaffold may include clusters of hexagonally arranged pores 502, as shown, for example, in FIG. 5C. If the round pores 502 are arranged so close that they overlap, they merge into one larger pore 504, illustrated in FIG. 5D, which has the shape of a hexagon, modified by convex edges. The larger pores may be advantageous in that they enable multiple neighboring cells to directly interact, yet provide contact guidance for cell alignment along the grooves of the pores. In certain embodiments, the microstructured scaffold is composed of two polymer materials, a harder polymer (such as, e.g., polycaprolactone) that forms the backbone defining the pores, and a softer polymer (e.g., hyaluronic acid) that fills the pores to provide additional support for the cells, akin to the extracellular matrix. During prolonged cell culture, the cells can digest the soft polymer, replacing it with their own excreted extracellular matrix.

In some embodiments, the distribution of pores is based on the anatomic distribution of photoreceptors in the retina. Typically, the rod density in the human retina is maximal in the center at 150000 rods/mm² and decreases towards the retinal periphery to 30000-40000 cells/mm². The rod diameter may increase from 3 μm in the area with the highest rod density to 5.5 μm in the periphery. The rods may be approximately 40 μm in length. Accordingly, a microstructured scaffold mimicking the retina may be about 40 μm thick, and feature a pore distribution that increases in pore size, but decrease in pore density, from the center towards the periphery. Further, to facilitate the physiological organization of both rods and cones, the scaffold may include different types of pores, tailored in terms of size, surface topography, and/or surface chemistry to rods and cones, respectively. The relative numbers of pores of each type may reflect the rod-to-cone ratio in the retina, which is approximately 20:1 at the location with the highest rod density.

In certain embodiments, the scaffold includes or essentially consists of a cage-like scaffold that holds cells in a centralized location, and has interfaces that dictate interaction with the RPE layer below and the neuroretina atop. The scaffold may be open to the retina on both sides, e.g., via pores, to allow for endogenous nutrients and regulators to enter the scaffold. In one embodiment, cells are typically within 200 μm of a nutrient source, due to many openings in the scaffold design for nutrient delivery. The scaffold may be constructed with a large pore size on one surface, typically the one facing the retinal neuroretina, and a smaller pore size on the other surface, which, accordingly, faces the RPE. Nutrients can diffuse in from the pigment epithelium through the smaller pores.

FIG. 6 depicts an exemplary cage-like scaffold 600. This scaffold contains multiple cages 602, each capable of containing a plurality of cells. The top surface of each cage contains several larger pores 604 that provide physical guidance for the orientation of the cells and interactions with the environment. The bottom surface contains smaller pores 606, which allow nutrient transport, but hinder epithelial cells from entering the scaffold. While the illustrated embodiment shows six round pores per cage, other pore shapes and configurations (e.g., hexagonal arrangements) may also be used. Further, in addition to or instead of guidance pores, geometric arrangement of posts may be incorporated into the cage to promote arrangement of cells into a correct, physiological formation. The cages may also be filled with a soft polymer mimicking the interphotoreceptor matrix.

The cage-like scaffold may be used in conjunction with a nanoporous, RPE-coated membrane and an artificial plexus layer to form a device such as, for example, the bioreactor 200 illustrated in FIG. 2A. Alternatively, the scaffold may be used independently as a cell delivery device. For example, it may be transplanted into a host retina in the layer 120 between the RPE and the outer limiting membrane, as indicated in FIG. 1.

A cage-like scaffold may, but need not, be fabricated in a layer-upon-layer method. Thus, each layer may be specifically designed to incorporate a number of topographical or biochemical cues. For example, each layer may contain microstructures, nanostructures, surface chemical patterning, and/or drug delivery mechanisms in hierarchical fashion to promote cell regulation during culture and after transplantation. Therapeutics can be incorporated into the scaffold by a number of means, including, for example, adsorption or conjugation (i.e., chemical bonding) to the surface, absorption into the bulk material, physical embedding or encapsulation, nanoparticle incorporation, or hydrogel delivery. By tailoring the design of a particular layer, the delivery of therapeutic molecules may be controlled to occur selectively towards the transplanted cells (modification of cage layer), neuroretina (modification of top layer), or the RPE (modification of bottom layer). Growth factors may be released into the cages holding the cells for pro-differentiation of the transplanted stem cells. Therapeutics delivered to the neuroretina include, for example, neuroprotective agents that limit further damage to remaining photoreceptors. Therapeutics delivered to the RPE and choriocapillaris may be utilized to modulate the degree of angiogenesis and neovascularization.

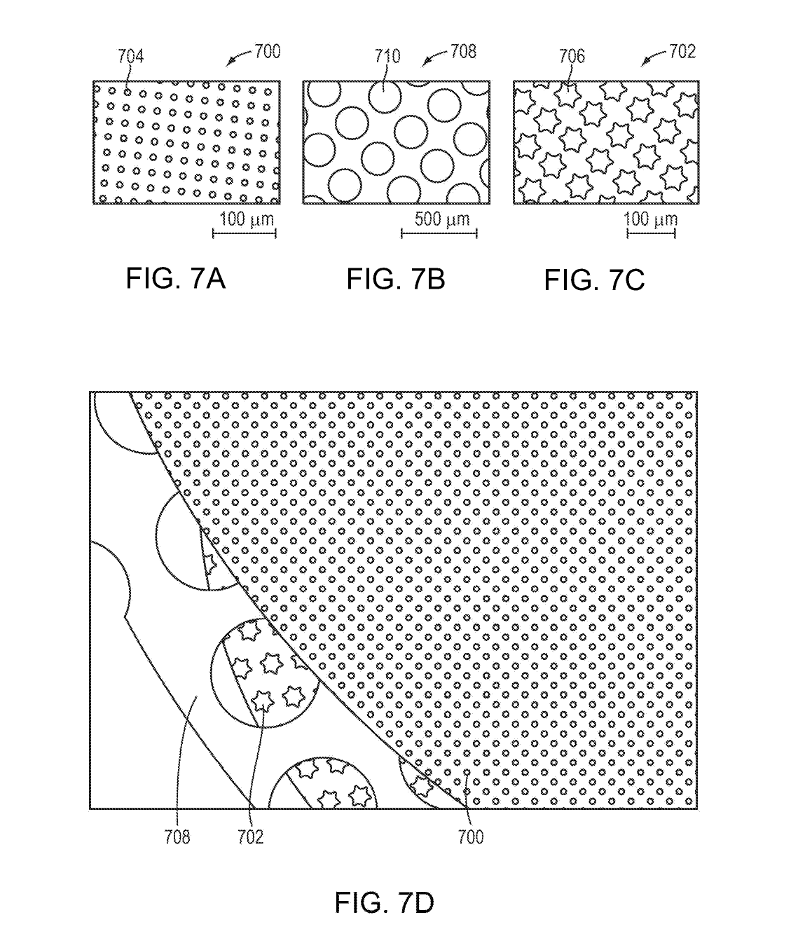

Each layer can be fabricated to any degree of thickness. For layer thicknesses between 1 and 20 μm, the final construct has a resulting thickness between 3 and 60 μm, which is sufficiently thin to generally not disturb the host retinal tissue. FIGS. 7A-7C illustrate the three layers of an exemplary cage-like scaffold separately. FIGS. 7A and 7C depict the bottom 700 (RPE-side) and top 702 (neuroretina-side) surface layer, respectively. Both layers include a regular lattice arrangement of pores 704, 706, albeit with different distance between adjacent pores. The bottom layer 700 features closely spaced circular pores 704 of about 10 μm in diameter, and the top layer 702 features more distantly spaced pores 706 of modified hexagonal shape with diameters of about 60 μm. The middle layer 708 includes circular openings 700 of about 200 μm in diameter, which define the side walls of the cages. (Note the difference in scales between FIGS. 7A-7C). FIG. 7D shows the three layers 700, 708, 702 stacked on top of one another.

### 2. Fabrication Methods

Bioreactor devices in accordance with embodiments of the invention may be manufactured using various techniques known in the art, including replica molding, conventional machining, injection molding, sacrificial molding, material printing, laser machining, or solid freeform fabrication. To produce the polymer scaffold and plexus by means of replica molding, a master mold featuring a negative relief of the desired structure is fabricated for each layer. Widely used methods of creating the master mold include soft lithography, wet etching, plasma etching, and electroplating.

Producing the master mold by soft lithography, for example, involves designing a photomask that defines the ridges of the master mold, corresponding to the indentations of the final layer, as transparent regions in an otherwise opaque sheet. The mask layout may be defined in a computer drawing, and may then be converted, e.g., with a software package such as Tanner L-Edit, into a Computer-Aided Design (CAD) layout, which is suitable for subsequent physical writing of the mask by electron-beam lithography or a similar technique.

As another preparatory step, a substrate wafer, e.g., made from silicon, may be spin-coated with a viscous solution of a suitable photoresist, such as, for example, SU-8. Typically, the wafer is spun rapidly, at 1200 to 4800 revolutions per minute, for a time duration ranging from several tens of seconds up to minutes, to produce a uniformly thick layer of photoresist with a thickness of up to tens or even hundreds of micrometers. Then, the photomask may be placed on the wafer, and the photoresist in the transparent regions of the mask may be chemically stabilized by exposure to UV light. Photoresist in non-exposed regions may be subsequently removed by exposure to a chemical developing agent, and the remaining photoresist may be hardened at elevated temperatures to form a durable negative relief. In an etching step, a chemical agent may be employed to remove the upmost layer of the substrate in regions that are not protected by photoresist, generating a channel pattern in negative relief in the wafer, which now constitutes the master mold. The photoresist, no longer needed, may afterwards be removed from the substrate. Next, a liquid polymer may be cast into the master mold, cured, and peeled off, resulting in a replica mold of the structured layer of the device (e.g., the scaffold 258, microchannel layer 202, 252, or individual layer of a cage-like scaffold 600).

The porous membrane that separates the plexus from the microstructured scaffold may be fabricated, for example, by coating a suitable material onto a wafer, curing it, if applicable, and peeling it off. Pores may then be mechanically punched into the membrane. Alternatively, the membrane may be lithographically patterned, like the other polymer layers. Another fabrication approach involves electrospinning of a membrane with desired porosity, thickness, and other desired properties. Alternatively, a commercially available membrane (e.g., a track-etched polycarbonate membrane from Sterlitech, Kent, Wash.) or a membrane fabricated in situ may be used.

In various embodiments, the inner and outer surfaces of the scaffold are micro- or nano-patterned. The patterning may be uniformly applied over the entire surface area, or selectively to certain portions of the surface area only. Surface patterning may be achieved during the fabrication of the polymer layer(s) that form(s) the scaffold, using techniques such as, for example, multi-layer photolithographic patterning, a combination of photolithography and etching, transfer molding, 3D printing, or flow lithography. Alternatively or additionally, surface patterning may be applied after completion of the polymer layer manufacturing process. For example, surface portions (e.g., the inner surfaces of pores in a particular region) may be chemically treated to modify their adhesive properties, conjugate therapeutic components to the surface, etc.

The layers (including, if applicable, the membrane) may then be stacked in the appropriate configuration, and bonded, e.g., via plasma treatment, chemical bonding, thermal bonding, or pressure bonding. For example, in the embodiments illustrated in FIGS. 2A and 2B, the artificial Bruch's membrane is sandwiched between the artificial plexus and the cell culture chamber or scaffold. The stacked layers may then be sterilized by conventional methods, connected to external microfluidic components and/or pressurizing components, and seeded.

Advantageously, the manufacturing techniques described above typically provide cost-effective, reproducible, and versatile means to fabricate three-dimensional scaffolds and bioreactor devices. Further, they generally provide a uniform and precise method for creating retinal scaffolds, and enable independent control of individual feature sizes and shapes. Thus, they enable designing structural components of the bioreactor devices so as to provide cues similar to those found in the developmental retina. For example, the micropatterned structures can be designed to influence initial cell attachment and spreading, and allow the maintenance of differentiated cell phenotype throughout the culture. In some embodiments, the ability to culture tissue with proper anatomical organization results in a greater chance for re-establishing photoreceptors in a reproducible manner.

### 3. Cell Culture and Tissue Engineering Methods and Applications

Cells may be seeded into the bioreactor devices or the scaffold utilizing conventional seeding techniques, backfilling, or encapsulation within a secondary gel matrix. Conventional seeding includes delivering the cells to the device by injection or flow, and allowing the cells to statically adhere. Backfilling includes the use of vacuum to disperse cells evenly. Encapsulation involves delivering the cells with a gel in order to distribute cells evenly in a three-dimensional matrix. In preferred embodiments, various cell-types are dynamically co-cultured. For example, RPE cells or stem cells seeded on the membrane may be cultured together with RPCs or stem cells that are contained in the scaffold or culture chamber. Further, the microchannels of the plexus layer, which are in communication with the membrane, may be lined with endothelial cells or stem cells, pericytes, or smooth muscle cells.

Cell co-cultures may provide biochemical cues that supplement the topographical and chemical patterning of the scaffold, thereby facilitating, and providing local control over, cell development and differentiation and tissue formation in the retina. For example, essential developmental and repair mechanisms may be accomplished by molecules secreted by RPE cells. RPE cells have been shown to synthesize, store, and/or secrete a number of trophic factors and cytokines, which may be essential for normal photoreceptor cell differentiation and regenerative repair. Cell differentiation may also be initiated by supplying neurotrophic factors directly to the cell culture medium. In one embodiment, the RPE in the culture chamber provides a chemical gradient for photoreceptor development along the depth of the chamber. However, chemical gradients may also be achieved by differential conjugation along the membrane surface, or by introduction of molecules through the plexus or microfluidic inlets or outlets in the scaffold.

In vivo, the retina and the RPE are dependent on a continuous dual supply of nutrients from the retinal and choroidal blood circulations. The bioreactor device described herein may be placed under continuous perfusion to maintain an effective oxygen and nutrient supply and, at the same time, effectively remove waste products in a manner similar to the native retina. In some embodiments, the dual compartment allows for polar application of test therapeutics and continuous analysis of culture medium on either the retinal or choroidal side. In various embodiments, physiological transport properties can be tailored to mimic that of the retina in vivo, which is generally not possible in conventional static cell culture systems.

Additional system components may readily be incorporated into the bioreactor design in order to stimulate or monitor cell culture. For example, microelectrodes may also be utilized to electrically stimulate retinal stem cells in a field-effect manner. Exemplary system components for monitoring the cell culture include electrodes that can be used to test for epithelial resistance and monolayer formation, biosensors for assessing pH and oxygen concentration, and continuous imaging and analysis to study the morphology of the cells and tissues. In one embodiment, these additions make the bioreactor suitable for automation, and improve the quality of the produced tissue.

In various embodiments, the bioreactor devices described herein recreate the architectural unit of the retina, and provide a dynamic in-vitro model of the retina with real-time functional analysis capabilities. As a result, they are suitable for the study of mechanisms of retinal damage and disease, including, e.g., toxic retinopathies and ocular neovascularization, as well as for the development of therapeutic methods. For example, the bioreactor devices may be employed in drug efficacy and safety testing. Advantageously, they allow for the testing of substances (such as, e.g., neuroprotective, antiangiogenic, or regenerative molecules) at the site of action in the posterior segment, and provide a tightly controlled experimental environment. The use of the bioreactor devices described herein may limit animal tests, and eliminate or reduce the need for complex tissue characterization techniques and assays.

The bioreactor devices and microstructured scaffolds may also be used in cell-based and transplant therapies. While previous methods for retinal stem cell or RPC delivery have not been able to differentiate sufficient numbers of photoreceptors for clinical application, various embodiments of the present invention provide physical, chemical, topographical, and physiological mechanisms that allow retinal stem cells to adopt positional identities and stable end-stage differentiation. By culturing stem cells in a dynamic environment similar to that found in retinal development, differentiation of stem cells to photoreceptors with outer segment-like structure can occur. Further, since cellular assembly and organization is foreseeable with the precise coordination of physical, spatial, biochemical, and physiological signals, the control of these parameters aids in standardizing RPC and stem cell culture in transplantable scaffolds and bioreactor devices in a reproducible manner.

In certain applications, multiple bioreactors may be coupled in parallel and operated as a single lot to provide increased lot sizes that achieve economies of scale. Alternatively, multiple bioreactors may be coupled in series for high-throughput screening applications. In comparison to traditional cell culture and tissue engineering methods, this limits the number of aseptic operations during the growth process from expansion to a final tissue product. At the end of the three-dimensional growth process, the tissue may be transplanted into a host retina. Alternatively, medium may be replaced by a cryopreservative solution, and the individual reactors may be sealed, utilizing the bioreactor itself as a component of sterile packaging. Combined with system components for automation, the bioreactor may provide a regulated end-stage retinal tissue product.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A bioreactor for culturing retinal cells or tissues, the bioreactor comprising:
a first polymer layer defining a network of microchannels;
a second polymer layer forming a scaffold; and
a porous thin-film membrane coated with retinal pigment epithelial cells on a surface facing the second polymer layer, the membrane separating the first polymer layer from the second polymer layer.

2. The bioreactor of claim 1, wherein pores of the membrane have diameters of less than 1 μm.

3. The bioreactor of claim 1, wherein the membrane comprises a polymer.

4. The bioreactor of claim 1, wherein a thickness of the membrane is in a range from about 2 μm to about 6 μm.

5. The bioreactor of claim 1, wherein the membrane is characterized by a diffusivity in a range from 200 μg/mm² per day to 300 μg/mm² per day.

6. The bioreactor of claim 1, wherein an inner surface of the scaffold is at least one of topographically or chemically patterned.

7. The bioreactor of claim 1, wherein the scaffold is microstructured so as to provide contact guidance for spatial cell organization.

8. The bioreactor of claim 1, further comprising at least one of retinal progenitor cells or stem cells seeded in the scaffold.

9. The bioreactor of claim 1, further comprising cell culture media in the scaffold.

10. The bioreactor of claim 1, further comprising means for perfusing at least one of the microchannel network or the scaffold.

11. The bioreactor of claim 1, further comprising means for controlling a pressure in at least one of the microchannel network or the scaffold.

12. The bioreactor of claim 1, wherein the network of microchannels forms an artificial plexus.

13. A bioreactor for culturing neuroretinal tissue, the bioreactor comprising:
a first polymer layer defining an artificial plexus;
a second polymer layer forming a microstructured scaffold; and
a porous thin-film polymer membrane separating the first polymer layer from the second polymer layer.

14. The bioreactor of claim 13, wherein the microstructured scaffold comprises polymer posts arranged at the vertices of a lattice.

15. The bioreactor of claim 13, wherein the microstructured scaffold comprises pores arranged at the vertices of a lattice.

16. The bioreactor of claim 15, wherein the pores have a modified hexagonal shape.

17. The bioreactor of claim 13, wherein the microstructured scaffold comprises clusters of hexagonally arranged pores.

18. The bioreactor of claim 13, wherein the microstructured scaffold comprises top, middle, and bottom layers, the top and bottom layers comprising through pores, the three layers together defining a cage structure for holding cells.

19. The bioreactor of claim 13, wherein the microstructured scaffold comprises a harder polymer structure forming pores filled with a softer polymer.

20. The bioreactor of claim 19, wherein the hard polymer comprises polycaprolactone and the soft polymer comprises hyaluronic acid.

21. In a bioreactor comprising a first polymer layer defining a network of microchannels, a second polymer layer forming a scaffold, and a membrane therebetween, a method of culturing retinal tissue, the method comprising:
(a) seeding at least one of retinal progenitor cells or stem cells in the scaffold;
(b) perfusing the network of microchannels with a fluid suitable for cell culture; and
(c) initiating cell differentiation of the retinal progenitor cells or stem cells.

22. The method of claim 21, further comprising seeding retinal pigment epithelial cells on a surface of the membrane facing the second polymer layer.

23. The method of claim 21, further comprising seeding vascular endothelial cells in the microchannels.

24. The method of claim 21, wherein initiating cell differentiation comprises supplying a neurotrophic factor.

25. The method of claim 21, further comprising transplanting the bioreactor into a patient's retina.

26. A cell-delivery device comprising:
a polymer scaffold defining at least one cage for housing cells, the scaffold featuring a first set of pores on a first surface thereof and a second set of pores, smaller than the pores of the first set, on a second surface thereof, the pores of the first and second sets being in fluidic communication with the at least one cage.

27. The device of claim 26, further comprising at least one of retinal cells, retinal progenitor cells, or stem cells housed within the at least one cage.

28. The device of claim 26, wherein the polymer scaffold comprises a first polymer layer defining side walls of the at least one cage, a second polymer layer defining the first set of pores and being bonded to the first polymer layer on a first surface thereof, and a third polymer layer defining the second set of pores and being bonded to the first polymer layer on a second surface thereof.

29. The device of claim 28, wherein the first, second, and third polymer layers each have a thickness in a range from about 1 μm to about 20 μm.

30. The device of claim 26, wherein the polymer scaffold comprises a biodegradable material.

31. The device of claim 26, wherein clusters of pores of the first set are hexagonally packed.

32. The device of claim 26, wherein the pores of the first set have diameters in a range from about 5 μm to about 70 μm, and the pores of the second set have diameters in a range from about 1 μm to about 20 μm.

33. The device of claim 32, wherein the at least one cage has a lateral diameter in a range from about 150 μm to about 300 μm.

34. The device of claim 26, wherein at least one of the first set of pores and the second set of pores are arranged at the vertices of a lattice.

35. The device of claim 26, wherein the polymer scaffold is implantable into a patient's retina and the pores permit ingress and egress of at least one of nutrients or regulators.

36. The device of claim 26, further comprising a soft polymer filling inside the at least one cage.

37. The device of claim 26, further comprising therapeutic molecules incorporated in the polymer scaffold.

38. A bioreactor device for mimicking the architecture of one or more layers of the retina, the device comprising a polymer-based scaffold structured at a micrometer or lower scale so as to facilitate physiologically functional spatial organization and assembly of cells seeded therein, and further comprising a microfluidic structure for supplying the cells with biochemical materials.

39. The bioreactor of claim 38, wherein the biochemical materials comprise oxygen and nutrients.

40. The bioreactor of claim 38, wherein the microfluidic structure facilitates transmission to the cells of biochemical or fluid-mechanical cues for cell differentiation.

41. The bioreactor of claim 38, wherein the polymer-based scaffold is topographically structured to facilitate physiologically functional spatial organization and assembly of cells seeded therein.

42. The bioreactor of claim 38, wherein the polymer-based scaffold is chemically structured to facilitate physiologically functional spatial organization and assembly of cells seeded therein.
